(19) United States
(12) Patent Application Publication
(10) Pub. No.: US 2014/0316792 A1
(43) Pub. Date: Oct. 23, 2014

(54) PROACTIVE AND PREVENTATIVE HEALTH CARE SYSTEM USING REMOTE MONITORING AND NOTIFICATIONS

(71) Applicant: eMobileCare Corporation, Redmond, WA (US)

(72) Inventor: Khalid Umar Siddiqui, Redmond, WA (US)

(73) Assignee: eMobileCare Corporation, Redmond, WA (US)

(21) Appl. No.: 14/210,099

(22) Filed: Mar. 13, 2014

Related U.S. Application Data
(60) Provisional application No. 61/802,674, filed on Mar. 17, 2013.

(51) Int. Cl. G06F 19/00; A61B 5/00; A61B 5/1455; A61B 5/0205; A61B 5/11

(52) U.S. Cl. CPC G06F 19/3418; A61B 5/02055; A61B 5/11; A61B 5/14551; A61B 5/6824; A61B 5/7435; A61B 5/746
USPC 705/2

(57) ABSTRACT

A health status monitoring and reporting device, system and method, comprising an user-wearable armband, a power source coupled with the armband, either or both of a positional sensor and a movement sensor coupled with the armband, a vital signs measurement device coupled with the armband, and a transmitter operably coupled with the power source, the transmitter being configured to receive from either or both of the accelerometer and the vital signs measuring device a signal including an indicator of a condition of the user, and further including a gateway device to transmit data from a ULP network to a Wide Area Network.

### RELATED APPLICATIONS

This application claims the benefit of priority to U.S. Provisional application No. 61/802,674, filed on Mar. 16, 2013, the entire contents of which are hereby incorporated herein by this reference.

### FIELD OF THE INVENTION

The invention relates generally to the field of health monitoring devices, and more particularly to monitoring the health condition status of a person via instrumentation and communication devices integrated in a patient-wearable structure.

### BACKGROUND OF THE INVENTION

Many people have loved ones living independently in the comfort of their own home. Their health and safety is a major concern.

The cost of healthcare is a critical issue in the United States, with nearly one of every five dollars' worth of the country's gross domestic product (GDP) going to medical expenditures. According to new data released by the research firm InMedica, the American tele-health market is predicted to grow by 600 percent between 2012 and 2017. While there are currently 227,000 tele-health patients in the United States, according to InMedica, that figure is forecast to reach up to 1.3 million patients in 2017. U.S. tele-health revenues, meanwhile, will jump from $174.5 million in 2012 to $707.9 million in 2017. InMedica defines telehealth as “the use of medical devices and communication technology to monitor chronic diseases and symptoms that could develop into serious conditions.”

Various types of patient monitoring systems are known. For example, U.S. Pat. No. 7,448,996 discloses a patient monitoring system for chronic diseases. U.S. Pat. No. 8,217,795 is for fall detection and alert. U.S. Pat. No. 7,453,364 is for simple measurement of activity and includes the safety of detecting if the device is removed from the body. The methodology used is to measure impedance by passing current through the body. U.S. Pat. No. 8,328,718 is monitoring physiological data.

Some of the known prior art devices and methods are intrusive due to sensors and wires strapped to the user's body, for example, which reduces the effectiveness of seamless monitoring. Others are meant as an alert mechanism and are not for monitoring or collecting useful data to provide proactive and preventive care, thereby providing limited reactive care only. Still others function in a manner that is inherently neither safe nor reliable, and in some cases raises privacy concerns.

Insofar as I am aware, no solution provides seamless and non-intrusive monitoring of physiological data, enabling the alerting of a critical situation and providing pro-active and preventive healthcare.

### BRIEF DESCRIPTION OF THE DRAWINGS

FIG. 1 shows a complete, end-to-end solution for independent living at home, according to an exemplary, non-exclusive embodiment.

FIG. 2 shows details of various components of the wearable device, according to an exemplary, non-exclusive embodiment.

FIG. 3 shows a process flow diagram of a design and development algorithm for the wearable device, according to an exemplary, non-exclusive embodiment.

FIG. 4 shows various configurations and types of the connections between the wearable device and the gateway, according to exemplary, non-exclusive embodiments.

FIG. 5 shows Gateway components to signify hardware in detail, according to an exemplary, non-exclusive embodiment.

FIG. 6 shows Gateway Services to signify software in detail, according to an exemplary, non-exclusive embodiment.

FIG. 7 shows a Cloud Computing Services Portal to illustrate various components and services, according to an exemplary, non-exclusive embodiment.

### DETAILED DESCRIPTION OF THE PREFERRED EMBODIMENTS

Health and safety of loved ones, living independently, is a major concern. A goal is to prolong independent living at home by providing proactive and preventive healthcare. This is done by remotely monitoring and storing physiological data and providing alerts and notifications. Family and other caregivers may view the current and historical data as a dashboard on their mobile devices at anytime from anywhere. Remote monitoring and alerting would provide proactive and preventive healthcare.

A wearable device, according to the disclosed embodiments of the invention, captures vital signs and critical situation data and send data and alerts to a cloud based portal. Extra care is taken to make the device non-intrusive and easy to use. The portal analyzes the collected data and displays results on a dashboard. Critical situations and trends are alerted to the caregiver and family members. Graphical reports are generated and allow caregivers to see trends. Reports also allow going back in time and analyzing events leading to an incident. The capture and analysis of data around-the-clock (24×7) forms a basis for proactive and preventive healthcare.

State of the art sensors and technology is used to seamlessly gather physiological data by using an unobtrusive arm band; the wearer won't even notice that it is there, most of the time. A remote monitoring service is alerted if a critical situation is detected, or if the device is taken off, or if it fails self-diagnostics. The device uses algorithms configured to enable ultra-low power consumption for collecting and sending data. Collected data is analyzed to provide proactive and preventive healthcare.

The invented embodiments include numerous beneficial features and functions, including for example:

1. A wearable device that is easy to use and non-intrusive, such that no sensor actually touches the body. This encourages user to wear it at all times.

2. A smart device running self-diagnostics to anticipate and report problem. The device also detects if user has removed it, and sends an alert. This is done by monitoring either or both of light reflected from the skin and the user's body temperature, or by the use of capacitive proximity detector which is triggered by the WD being within close proximity to the user's skin surface (e.g., upper arm, etc.).

3. Data is captured from environmental sensors, for example a door open/close sensor or gas/water/electricity usage sensors, and provides a complete picture of a user's activities to caregiver or the user's family.

4. Caregivers and family can connect to the dashboard of the portal, and can view whether a current status is all ‘green;’ e.g., in a normal situation. If a critical situation is detected, an icon turns red and an alert and notifications are sent out. The portal also produces and provides graphical reports for caregivers to view trends. In case of an incident, historical data leading to the incident may be viewed and analyzed.

5. Data are stored in the cloud and analyzed for trends and patterns to provide proactive and preventive care. Such data provide valuable insight to the family, doctor, and other caregivers, to modify/adjust a treatment protocol to improve health and prevent incidents leading to critical situations.

6. The invented device, system and method provides visibility to family members by showing health and safety related data in an easy-to-understand graphical and dashboard format. The family stays in touch and informed of any critical situation by receiving alerts and notifications on their existing mobile devices.

7. The device and the gateway are plug-and-play. The user plugs the gateway into a power outlet, the gateway auto-connects and authenticates to the web service portal. The user then touches the WD to the gateway to authenticate and connect. There is no setting or configuration needed.

8. The wearable device includes coded instructions embodying an algorithm which, when executed on the device, cause the device's operation to consume an ultra-low amount of power. The battery will last several weeks between charges.

An upper arm band is worn to capture and transmit data related to the health and well-being of the wearer. In case of a critical situation, alerts and notifications are sent to caregivers and registered family members. User may also request assistance by tapping at the arm band. The web service also analyzes data for deviations from normal status and measurements, and for specific patterns. Alerts and notifications are sent to caregivers if anomalies are detected. This provides proactive and preventive care for better health and well-being. A big advantage is the lowering of the healthcare costs.

One embodiment of the invention relates to a solution to capture physiological and environmental data. Physiological data includes vital signs and body movement patterns. Environmental data includes sensors to detect opening and closing of doors, ambient temperature, gas or water leaks, sound, motion, read Near Field Communications (NFC) tags or Radio Frequency Identification (RFID) tags, and light detection. Such data help in forming a complete picture of a user's status, environment, and activities, for making informed decisions for healthcare and safety.

The embodiments depicted in the several drawing figures have several hardware and software components, as are described in detail in the following paragraphs. For convenient reference, the numeric labels in the drawing figures correspond to the following features:

Drawings - Reference Numerals

| | | | |
|---|---|---|---|
| 100 | Upper arm band as a Wearable | 102 | Ultra-Low Power (ULP) Network |
| | Device (WD) | | using Bluetooth 4.0 or Zigbee IEEE |
| | | | 802.15.4 |
| 104 | Environmental sensors like door | 106 | Gateway device to connect ULP |
| | open, gas detected, water, | | network to Wide Area Network |
| | temperature etc. | | (WAN) |
| 108 | Cellular transceiver | 110 | WI-FI transceiver |
| 112 | Cloud based services | 114 | Web Portal for dashboard and data |
| 116 | Database in the cloud | 118 | Range of various mobile devices |
| 200 | Rechargeable 3.3 volt battery | 202 | Accelerometer MEMS chip |
| 204 | Reflectance Heart Rate Monitor/ | 206 | Pressure Sensor/Altimeter MEMS |
| | Oximeter MEMS chip | | chip |
| 208 | Gyroscope MEMS chip | 210 | Infrared Temperature sensor chip |
| 211 | Proximity Sensor (Capacitive) | | |
| 212 | Self-Diagnostics logic | 214 | System on Chip with ULP Radio |
| 216 | Near-Field Communication | 500 | Bluetooth 4.0/802.15.4 |
| | (NFC)/Radio-frequency | | transceiver |
| | identification (RFID) Tag | | |
| 502 | Microphone/Speaker | 504 | Web Cam |
| 506 | 2 Way Communication Hub | 508 | Wide Area Network Interface |
| 510 | Cellular radio/transceiver | 512 | Wi-Fi radio/transceiver |
| 514 | Battery + Mains Power Supply | 516 | Buttons to clear Alarm & |
| | | | Reminders |
| 518 | NFC/RFID Reader | 520 | GPS (Optional) |
| 522 | LCD Screen (Optional) | 600 | Admin task software module |
| 602 | Security and Compression module | 604 | Network Provider module |
| 606 | Alarm/Reminder provider | 608 | Cloud data provider |
| 610 | Watchdog provider | 612 | Database SQL Lite |
| 614 | Content Provider | 616 | Self & Network diagnostics |
| 618 | Simulated Data UI | 620 | Device Data provider |
| 700 | API's for data transfer | 702 | Database engine at the portal |
| 704 | Admin Tasks at the portal | 706 | Reminders engine |
| 703 | Reports engine | 710 | Portal Dashboard |
| 712 | E-Mail engine | 714 | SMS Text message engine |
| 716 | Care Giver mobile devices | | |

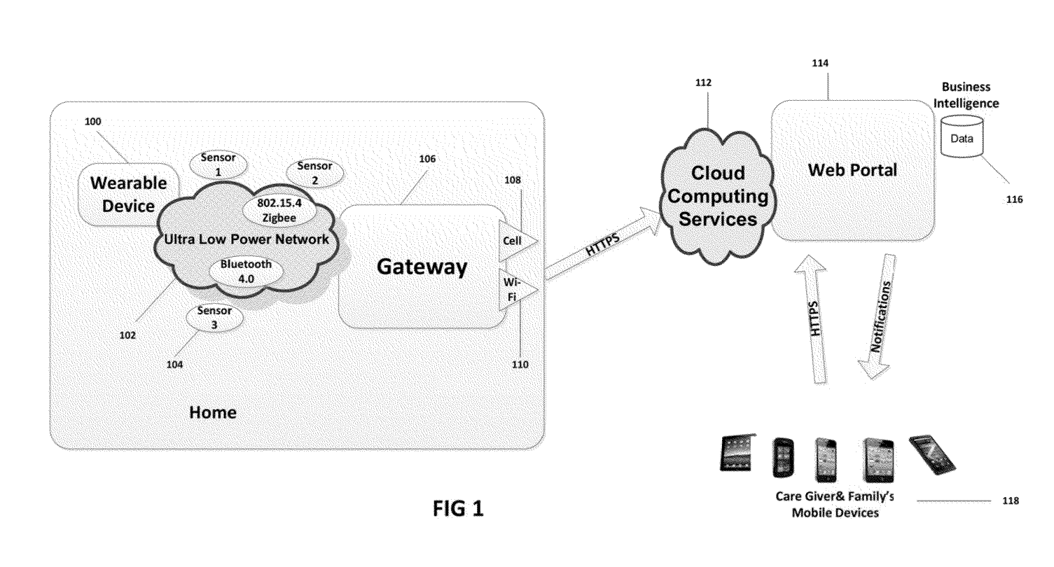

FIG. 1 shows tae complete end-to-end solution for independent living at home according to an exemplary, non-exclusive embodiment. The wearable device (WD) is an arm band 100 with sensors and electronics to capture and transmit useful data. The data are transmitted via an Ultra-Low Power Network (ULTP) 102. The ULTP is based on two low power protocols; Bluetooth 4.0 (Low Energy), and IEEE 802.15.4 (based on the Zigbee communication protocols and specification, for example). The design is modular to allow use of either of the protocols, or a mix of both. For example, the WD provides good results with Bluetooth 4.0, and environmental sensors work better with Zigbee.

In another embodiment, an ANT protocol is used, which is similar to Bluetooth 4.0. The Gateway 106 is connected to the ULTP 102 with built-in transceivers for Bluetooth 4.0 and 802.15.4 protocol-based networks. An environmental sensor 104 connects to the gateway 106 via one or the other of the ULTP protocols. The gateway 106 connects to the internet in order to access Cloud Computing Services 112, using a wide area network interface, for example the cellular radio 108.

In one embodiment, the cellular network used is 3G (as provided by AT&T or T-Mobile, for example). In another embodiment, the cellular network used is CDMA (as provided by Sprint or Verizon, for example).

The gateway 106 can also connect to the internet by using the built-in Wi-Fi interface 110. This method may only be used if a Wi-Fi router is available and functional at the place where the user will be monitored; e.g., at the place of residence. Sending data over Wi-Fi is cost effective. A built-in, device-executable instruction set embodies an algorithm configured to prefer sending data to the web service via Wi-Fi, if and when available.

The connection to the Cloud Computing services is encrypted and compressed by using HTTPS and JSON protocols (routinely used by banks and financial services). The Web Portal 114 is hosted by a cloud service provider known as ‘Platform as a Service’ (PaaS). In one embodiment, Amazon Web Service (AWS) is used for hosting the Web Portal 114 and the associated Database 116. In another embodiment, Windows Azure is used as PaaS. In yet another embodiment, Google App Engine is used. In all cases, an associated Database engine 116 is used to store persistent data.

The Web Portal 114, provides data access to registered caregivers and family member in graphical and text format. The caregivers and family members may avail a wide range of existing mobile devices 118 to monitor the current situation, as well as to receive alerts via SMS and e-mail notifications, includes iPhone, iPad, Android phones/tablets, Windows phone/tablets/personal computers, etc. The only requirement is that the mobile device should have data and text access to the internet.

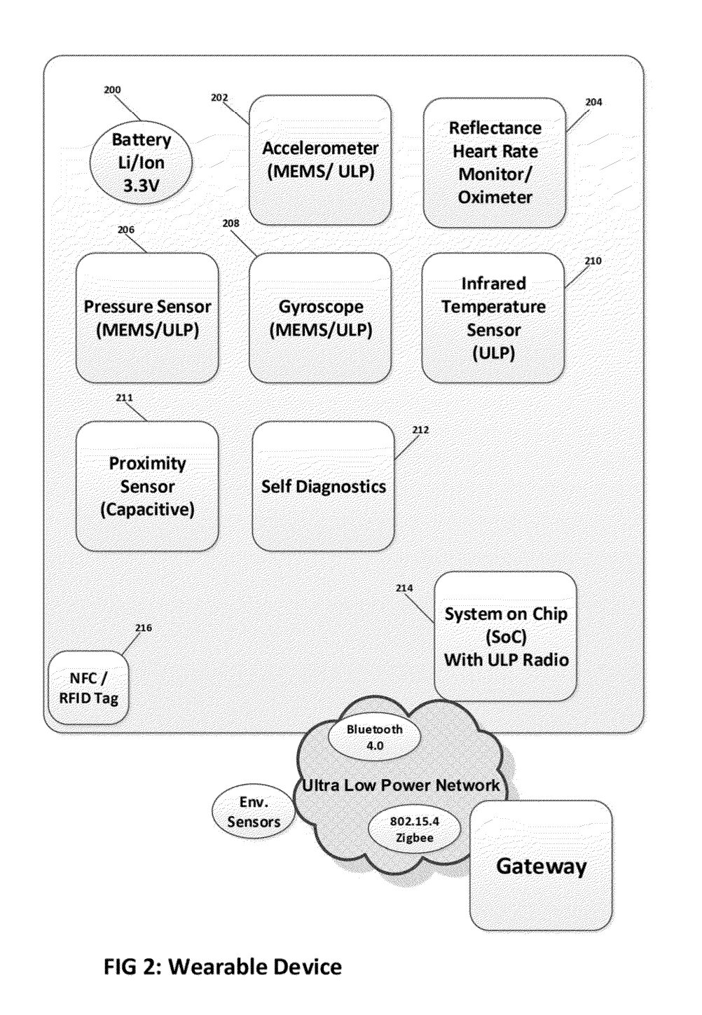

FIG. 2 depicts a block structural diagram of an embodiment of the Wearable Device (WD) configured, for example, as a watch size device attached to an arm band. The WD is sufficiently comfortable and non-intrusive that a user is likely to forget that it is even being worn.

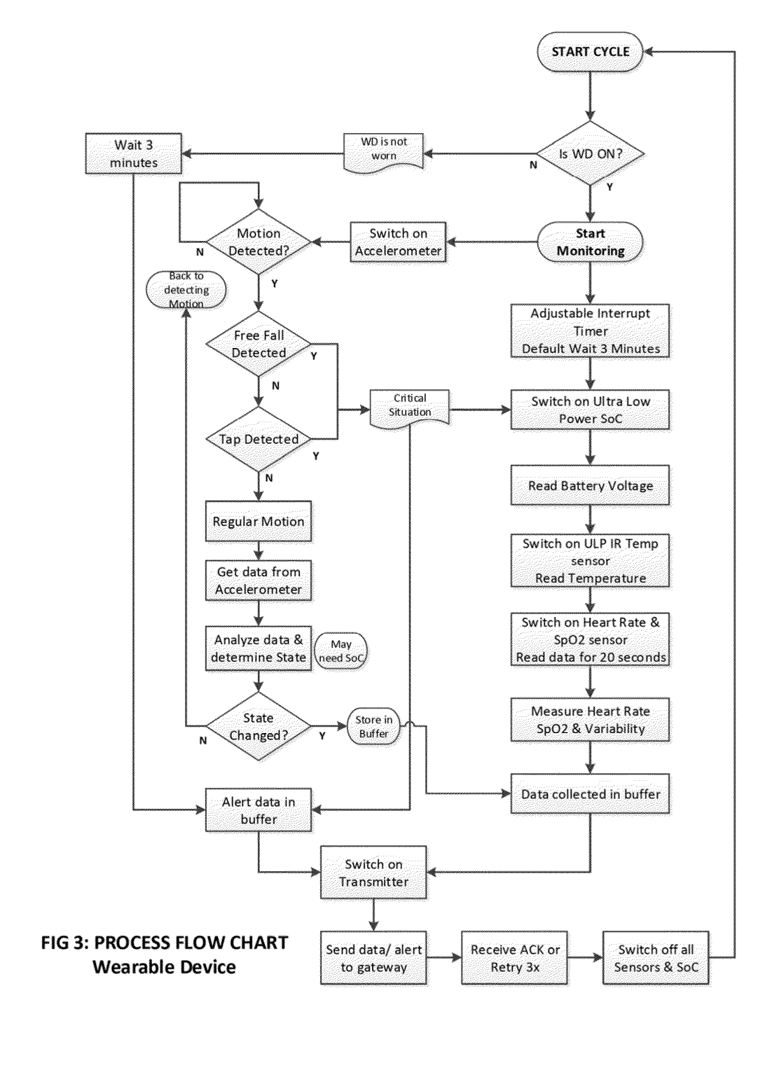

The power is supplied by a 3.3 volts Lithium Ion rechargeable battery 200, for example, although other battery technologies that likewise provide a suitable voltage are also contemplated within the embodiments. In a preferred embodiment, the WD is water proof and consumes an ultra-low amount of power, therefore the battery is expected to last several months to a year between charges. A special algorithm (as in the exemplary embodiment of FIG. 3) is embodied in device-executable instructions configured, when executed on processing circuitry of the device, to ensure a low power consuming mode of operation.

A user's body motion is detected by an ultra-low power Accelerometer 202 on a Micro-Electromechanical System (MEMS) chip. In an exemplary embodiment, the accelerometer chip is an Analog Devices ADX.L362 device, and the Gyroscope 208 is an InvenSense IMU 3000 device. In another exemplary embodiment, an InvenSense MPU9250 chip combines each of an Accelerometer 202, a Gyroscope 208, and a Pressure/Altitude sensor 206 in a single MEMS chip.

In exemplary embodiments, the Digital Infrared temperature sensor comprises a Texas Instruments TMP006 210 device, and the Pressure/Altitude sensor is a Freescale MPL3115A2 206 device. The Reflectance Heart Rate Monitor/Oximeter 204 is a subassembly containing an Infrared LED, a red LED, a photo sensor, and a Texas Instruments AFE4490 analog front end device with operational amplifiers and filters, to obtain heart rate and oxygen saturation level by measuring the light reflected by the user's arm. Such configuration makes the WD non-intrusive and safe, with none of the sensors actually touching the skin.

A System-on-Chip (SoC) 214 is a microprocessor based system with all the necessary components combined on a single chip, including an ultra-low power transceiver for connection to the gateway. In one embodiment the SoC is a Texas Instruments CC2541 device, which has a built-in Bluetooth 4.0 transceiver. In another embodiment, the SoC is an Atmel ATMEGA128RFA1 device, which has a built-in Zigbee transceiver. In yet another embodiment, the SoC is a Nordic nRF51822 device, which has a built-in Bluetooth 4.0 transceiver. In an embodiment, an I2C bus and an SPI bus are used to connect sensors to the SoC, as all sensors have either a I2C bus or SPI bus connection capabilities. In an embodiment, a Reflectance Heart Rate Monitor/Oximeter is connected to an analog-to-digital converter (ADC) of the SoC. In still another embodiment, the analog-to-digital conversion is performed on the subassembly, and only the result is fed to the SoC.

The WD typically includes an NFC/RFID tag for identification, which is read by an environmental sensor for tracking and also for identification to the gateway. The WD is authenticated and paired to the gateway by bringing it to within a close proximity to the gateway.

FIG. 3 shows a Process Flow Chart for the Wearable Device according to an exemplary embodiment, depicting a software structure to enable software development by an ordinarily skilled artisan. FIG. 3 shows the algorithm used for power management and self-diagnostics of the device. At the top level, WD being worn is detected by the proximity detector. If WD is not being worn, an alert is sent and the cycle is terminated (as shown in FIG. 3), thereby saving power. The SoC and all sensors (except the accelerometer) are either off or in sleep mode most of the time. The ultra-low power accelerometer is on at all times, and it raises (or ‘generates’) interrupts in case of a critical situation like a fall or a tap. Tapping on the device, which is detected by the accelerometer, indicates a request for help in the manner of a ‘panic button.’

Detection of a critical situation by the accelerometer interrupts wakes up a Microprocessor unit (MCU) in the SoC to perform further analysis, and to send the data and an alert to the gateway using the ultra-low power network. The accelerometer also detects normal motion and categorizes the type of motion, and sends the data to the gateway. Every five minutes, or at another programmable interval, the SoC and the sensors are woken up to obtain readings of vital signs, even if there is no motion detected. Self-diagnostics, such as reading a battery voltage level and a check to see if the device is being worn, are performed at this time and data is sent to the gateway.

A device-being-worn status is checked by measuring two parameters. A first parameter is from the heart rate monitor's reflectance of light from the skin of the arm. If no light is reflected, the device is likely to be off the arm (not being worn). A second parameter is determined by detecting the skin temperature, which should be higher than the ambient temperature. With these two parameters, a decision could be made whether or not the device is being worn. The status is then sent to the gateway for further processing. Alternatively, the status is checked by the capacitive proximity detector. If the device is not being worn, the measurement cycle would terminate at the start to conserve the usage of power. The WD also supports upgrade of firmware ‘over the air,’ if initiated by the gateway and/or cloud computing service.

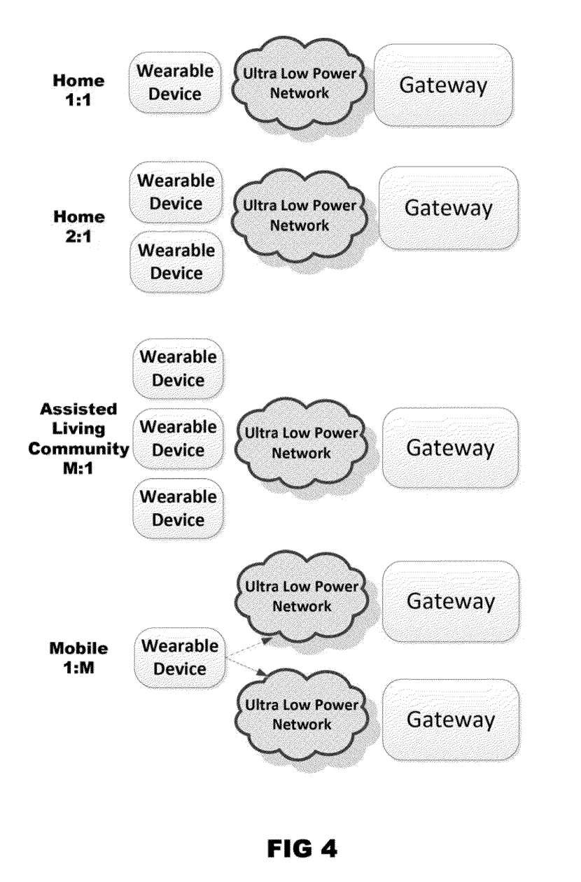

FIG. 4 shows connection combinations between the wearable device and the gateway in different situations. A 1:1 (one-to-one) connection is for a single person living at home. A 2:1 (two-to-one) connection is for two people living together at home. A single gateway is shared between the two devices. Typically, each fragment of data sent by the WD has a device ID in the header, enabling the gateway to establish the source of the data. A M:1 (many-to-one), or multiple WD to one gateway, is an extension of the 2:1 connection and works on the same principal. This scenario applies to assisted living and retirement home communities. A 1:M (one-to-many) is when the user is mobile and moves from one gateway to another based on location. Examples include, a WD user moving from a room to a community gathering area within an assisted living or retirement home, or one WD user visiting another WD user. The common theme is that a WD user can utilize any allowed gateway, using the gateway as a data conduit to the cloud computing service. However, downstream services like a medicine reminder and two-way communication would typically only work with the assigned gateway

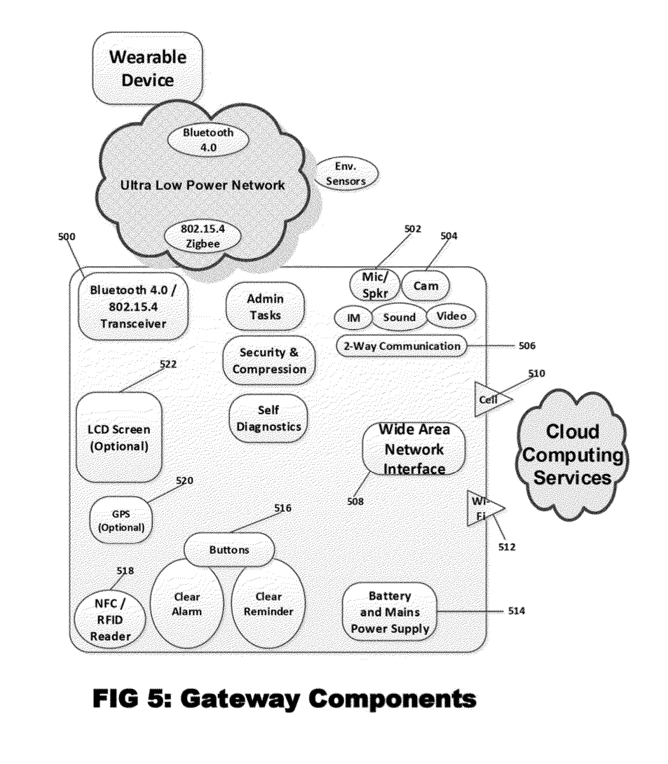

FIG. 5 shows the component view of the gateway. The gateway comprises electronics hardware and a software device that connects an ultra-low power network of the WD and sensors to the wide area network of Cloud Computing Services. In essence, it is a hub of the ultra-low power network by using a set of Bluetooth 4.0 and 802.15.4 Transceivers 500. A connection to a wide area network is provided by using the Cellular radio 510 and Wi-Fi module 512. The Wide Area Network Interface 508 has a software component to maintain and prioritize connection to wide area network.

Power to the gateway is supplied by a rechargeable battery with 8 to 10 hours life and an external mains charger 514 to keep the battery charged while plugged into AC power. The gateway allows 2-way communications with the authenticated caregivers 506. A camcorder 504 for video, and a microphone/speaker 502, facilitate communication. Instant messaging is also supported, in case sound or video is not convenient. Optional LCD screen 522 also facilitates communication.

There are modules for administration tasks, to provide security, authentication and data compression. A self-diagnostics module monitors and reports hardware, software and networking issues. Hardware and software buttons 516 allow the user to clear reminders and alarms. RFID tag 518 is used for identification and for tracking the location of the gateway. An optional GPS module 520 is used for tracking the exact location, if needed.

An embodiment of the gateway is implemented by using a modified tablet computing device (e.g., based on the GOOGLE ANDROID operating system, or an APPLE IPAD device) with a custom application/software (e.g., an “App”). Yet another embodiment of the gateway is implemented by using custom hardware and custom software.

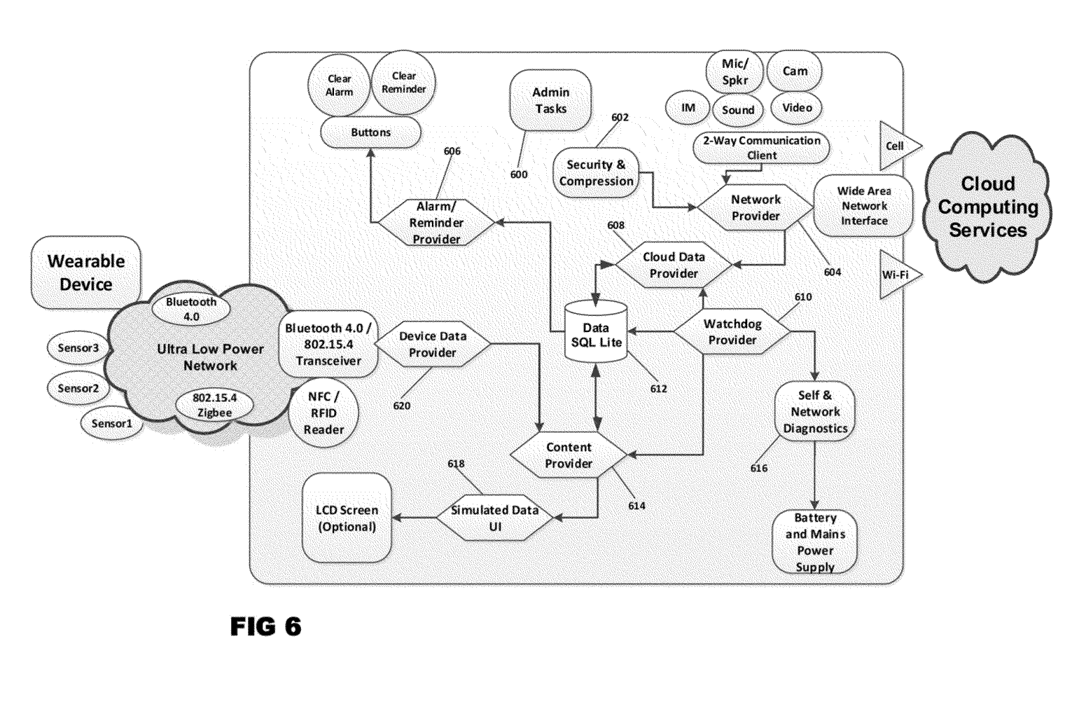

FIG. 6 shows an embodiment of the software components, functions, and relationships. Administration tasks module 600 supervises the operation and administration. Security and Compression module 602 provides authentication (device-to-gateway, portal-to-gateway, and users-to-gateway), authorization, and compression. A Security and Compression module 602 works closely with the Network Provider module 604. Network provider 604 interacts with the Wide Area Network interface to facilitate data flow and formatting while maintaining security. Network Provider 604 works with a two-way communication client (e.g., a SKYPE client), and also exchange data with Cloud Data provider 608.

The data is persisted (e.g., stored) in SQL Lite database 612. Device Data provider 620 collects data from the WD and other sensors, and passes the data to Content provider 614. Content provider stores the data in a SQL Lite database. Watchdog provider 610 is a background service that monitors data flow and persistence. Data is stored in the database till the successful transfer takes place, and then is deleted. Simulated Data UI 618 is optional for testing, and displays data transfer. Network and Self Diagnostics module 616 provides diagnostics for battery, hardware, and network traffic, and send alerts if needed. Alarms and Reminder provider 606 monitors alarms and reminders from the database and provides visual and sound notification. The notification may be cleared by the user by pressing a hardware or touchscreen button, which is also recorded and sent to the portal.

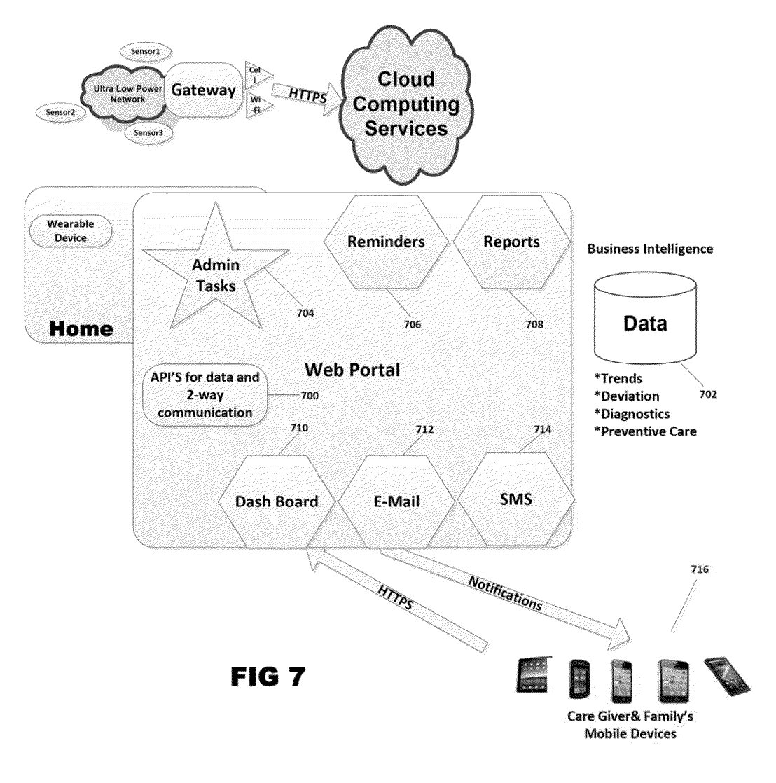

FIG. 7 shows the Cloud Computing Services (or ‘web’) portal. In an exemplary embodiment, the web portal is built using Django 1.4 and Python utilities hosted on Apache 2 at Amazon Web Services. A set of Application Programming Interfaces (API) 700 allows data transfer to and from the gateway and allows the two-way communication. The database service is provided by Amazon Relational Data Service (RDS) in the cloud, in an embodiment, based on MySQL database engine 702.

A business intelligence type process is run every three hours (or at some other time interval) to analyze trends, patterns, and deviations from normal conditions, enabling the provision of proactive and preventive care. The Administration task 704 is provided by the admin console, allowing tasks such as creating users, groups, and access levels. The database 702 stores user identification, login credentials, WD data, sensor data, user profiles, data access permissions, notifications and alerts, and reminders.

Reminders engine 706 gathers and transfer to the gateway the data associated with the user. Reports engine 708 gathers physiological and sensor data per user and provides graphical reports to the dashboard. Dashboard engine 710 is the primary interface to caregivers and family associated with a user after authentication. Caregivers and family members may connect to the portal from their mobile devices 716 at anytime and from anywhere in the world over the internet. IPHONE, IPAD, WINDOWS phone, ANDROID phone and tablet user can load the native application for a better experience. All other users can use a web browser to access the portal. Text Notifications are sent by SMS engine 714 based on the an SMS texting application (e.g., as is available from TWILLIO, etc.) in an embodiment. E-Mail notifications are sent by E-Mail engine 212 using SMTP as provided by AWS, in an embodiment.

Operation

The WD operation is very simple. The user just wears it on the upper arm and forgets about it. In case of a critical situation, WD will automatically detect and send an alert. However, the user can alternatively summon help by tapping the WD with their finger. This is equivalent to pressing the ‘panic button.’

The gateway is plug-and-play; it needs to be plugged in to AC power and switched on. At the first use, WD is authenticated, paired and bonded to the gateway, by touching the WD to the gateway. This is accomplished by the use of NFC. The gateway is typically pre-configured to auto-connect to the WD and environmental sensors, and will also establish connection to a Cloud Computing Services Portal. Optionally, the gateway could be connected to a Wi-Fi router, if a router is available. If a critical situation is detected, or is indicated by a tap, the gateway will beep and a ‘Clear Alarm’ button will flash. User may cancel the alert by pressing or touching the Clear Alarm button on the gateway. If a reminder is set at the portal, for example, to take medicine, the gateway will beep and announce the reminder. The user may cancel the reminder by pressing or touching a ‘Clear Reminder’ button. Alternatively, the reminder alert will auto-stop in 30 seconds (or upon the expiration of some other predetermined time duration).

The caregiver or family member may download the native application to their existing mobile device (if supported). The application will invite them to log in by providing credentials (first time only). They will then have access to the dashboard on the portal, and can view the current status, and run and view graphical reports. Normally the status is all green (e.g., no critical situation or fault condition present). In case an alert is generated, the corresponding icon will turn red and the caregiver or family member will receive an SMS text, followed by an e-mail message. The caregiver or family member can also clear the alert from the portal. The caregiver or family member can also set a recurring or one-time reminder from the portal. The text and e-mail is received, even if they are not connected to the portal.

The caregiver or the family member may be tied to more than one user. In this case they will be able to view the dashboards of all the users they are authorized to view.

### CONCLUSION

A preventive and proactive healthcare system is described. This is accomplished by using a wearable device to monitor vital signs and critical situation. The data is transferred on a 24×7 basis to cloud computing services for analysis. The WD automatically raises an alert if a critical situation is detected. The alert is received by the portal, which will alert caregivers and family members. The alert will repeat every five minutes (or some other selected time interval) until the alert is cancelled. The cloud-based data is auto-analyzed at a frequent interval, looking for deviation from the norm. If a deviation is found, an alert is raised. A family doctor may be notified, to facilitate in providing proactive and preventive care. This may also indicate a need to change or modify dosage of prescribed medicines.

The captured data may also be used like the ‘black box’ in an aircraft. In case of an incident, a caregiver and a doctor look back in time via the data to see what happened a few hours, a day, a week or a month before the incident, and draw conclusions. With the right data, tools, and studies, this will have a huge impact on the quality of healthcare and will result in cost savings.

It will be understood that the present invention is not limited to the method or detail of construction, fabrication, material, application or use described and illustrated herein. Indeed, any suitable variation of fabrication, use, or application is contemplated as an alternative embodiment, and thus is within the spirit and scope, of the invention.

It is further intended that any other embodiments of the present invention that result from any changes in application or method of use or operation, configuration, method of manufacture, shape, size, or material, which are not specified within the detailed written description or illustrations contained herein yet would be understood by one skilled in the art, are within the scope of the present invention.

Finally, those of skill in the art will appreciate that the invented method, system and apparatus described and illustrated herein may be implemented in software, firmware or hardware, or any suitable combination thereof. Preferably, the method system and apparatus are implemented in a combination of the three, for purposes of low cost and flexibility. Thus, those of skill in the art will appreciate that embodiments of the methods and system of the invention may be implemented by a computer or microprocessor process in which instructions are executed, the instructions being stored for execution on a computer-readable medium and being executed by any suitable instruction processor.

Accordingly, while the present invention has been shown and described with reference to the foregoing embodiments of the invented apparatus, it will be apparent to those skilled in the art that other changes in form and detail may be made therein without departing from the spirit and scope of the invention as defined in the appended claims.

I claim:

1. A health status monitoring and reporting device, comprising:
an armband suitably configured to be worn on a user's arm;
a power source coupled with the armband;
either or both of a positional sensor and a movement sensor coupled with the armband;
a vital signs measurement device coupled with the armband; and
a transmitter operably coupled with the power source, the transmitter being configured to receive from one or more of positional sensor, the movement sensor, and the vital signs measuring device a signal including an indicator of a condition of the user.

2. The health status monitoring and reporting device of claim 1, wherein the vital signs measurement device is either or both of a reflectance heart rate monitor and a reflectance oximeter.

3. The health status monitoring and reporting device of claim 2, wherein the vital signs measurement device is a Micro-Electromechanical System (MEMS) device.

4. The health status monitoring and reporting device of claim 1, wherein the positional sensor is a gyroscope.

5. The health status monitoring and reporting device of claim 1, wherein the movement sensor is an accelerometer.

6. The health status monitoring and reporting device of claim 1, wherein either of both of the positional sensor and the movement sensor is a Micro-Electromechanical System (MEMS) device.

7. The health status monitoring and reporting device of claim 1, wherein any two or more of the transmitter, the vital signs measurement device, the positional sensor and the movement sensor are configured as part of a System-on-Chip device.

8. A health status monitoring and reporting system, comprising:
an armband including operably coupled monitoring and reporting devices including:
a power source,
a vital signs measurement device,
one or more sensors selected from the group consisting of a positional sensor, a movement sensor, and a pressure sensor, and
a transmitter coupled with the power source and configured to transmit from the armband to an ultra-low power network (ULPN) data received from one or more of the vital signs measurement device, the positional sensor, the movement sensor and the pressure sensor; and
a gateway device to transmit the data from the ULPN to a Wide Area Network (WAN).

9. The health status monitoring and reporting system of claim 8, wherein the transmitter is configured to communicate with the ULP via either or both of low energy Bluetooth and IEEE 802.15.4 communication technologies.

10. The health status monitoring and reporting system of claim 8, wherein the transmitter is further configured as a wireless signal transceiver.

11. The health status monitoring and reporting system of claim 8, wherein the vital signs measurement device is either or both of a reflectance heart rate monitor and a reflectance oximeter.

12. The health status monitoring and reporting system of claim 8, wherein the gateway device is configured to transmit the data to the WAN via either or both of cellular and Wi-Fi communication technologies.

13. The health status monitoring and reporting system of claim 8, wherein:
the positional sensor is a gyroscope,
the movement sensor is an accelerometer, and
the pressure sensor is an altimeter.

14. The health status monitoring and reporting system of claim 8, wherein any two or more of the transmitter, the vital signs measurement device, and the one or more sensors are configured as part of a System-on-Chip device.

15. A health status monitoring and reporting method, comprising:
providing to a caregiver a patient-wearable monitoring device comprising:
an armband;
a power source coupled with the armband,
a vital signs measurement device coupled with the armband,
one or more sensors coupled with the armband, wherein the one or more sensors are selected from the group consisting of a positional sensor, a movement sensor, and a pressure sensor, and
a transmitter coupled with the power source and configured to transmit from patient-wearable monitoring device to an ultra-low power network (ULPN) data received from one or more of the vital signs measurement device, the positional sensor, the movement sensor and the pressure sensor;
storing at a non-transitory data storage medium data including a unique identification corresponding to the patient-wearable monitoring device.

16. The health status monitoring and reporting method of claim 15, further comprising:
storing at a non-transitory data storage medium data including one or more selected from the group consisting of a user identification, login credentials, patient-wearable monitoring device data, sensor-captured data, user profiles, data access permissions, notifications/alerts, and reminders.
